(19) United States
(12) Patent Application Publication
(10) Pub. No.: US 2026/0263074 A1
(43) Pub. Date: Sep. 10, 2026

(54) SYSTEMS AND METHODS FOR EMBOLIZING BLOOD VESSELS

(71) Applicant: THE JOHNS HOPKINS UNIVERSITY, Baltimore, MD (US)

(72) Inventors: Prateek C. GOWDA, Baltimore, MD (US); Clifford R. Weiss, Baltimore, MD (US)

(21) Appl. No.: 18/873,548

(22) PCT Filed: Jun. 8, 2023
(86) PCT No.: PCT/US2023/024827

§ 371 Date: Dec. 10, 2024

Related U.S. Application Data
(60) Provisional application No. 63/351,034, filed on Jun. 10, 2022.

(51) Int. Cl. A61B 17/12; A61B 17/00

(52) U.S. Cl. CPC A61B 17/12031; A61B 17/12109; A61B 17/12136; A61B 2017/00022; A61B 2017/00123; A61B 2017/00128; A61B 2017/00199; A61B 2017/1205

(57) ABSTRACT

A system includes an assembly that is at least partially insertable into a patient. The assembly includes a catheter that is insertable into an artery. The catheter has an outlet. The catheter is configured to receive therethrough an embolic agent and deliver the embolic agent through the outlet. An occlusion device is coupled to the catheter. The occlusion device is configured to fully occlude the artery. A pressure sensor is configured to determine a pressure within the artery at a location proximal of the occlusion device.

### CROSS-REFERENCE TO RELATED APPLICATION

This application claims priority to and the benefit of the filing date of U.S. Provisional Patent Application No. 63/351,034, filed Jun. 10, 2022, the entirety of which is hereby incorporated by reference herein.

### FIELD

This application relates to systems and methods for embolizing blood vessels.

### BACKGROUND

It can be advantageous to occlude a blood vessel such as an artery. As one example, hepatocellular carcinoma (HCC) is the most common primary malignant disease of the liver and the third leading cause of cancer deaths worldwide, accounting for 600,000 deaths globally each year. 85% of HCC cases are diagnosed at an advanced stage at which the tumor cannot be surgically resected. Transarterial embolization (TAE) therapy is currently the primary alternative to surgery. TAE involves deploying a microcatheter to transvascularly inject drug and/or embolic agents at the tumor site. This procedure induces ischemic necrosis of the tumor by restricting blood flow. TAE was established as a standard of care for intermediate/late-stage HCC in 2006 and it is currently the primary treatment modality for nonresectable cases. Still, there are currently no objective techniques to determine the optimal endpoint for the procedure, resulting in variable tumor responses. While under-embolization can result in inadequate management of the targeted tumor, over-embolization can result in off-target embolic tissue delivery, which increases liver toxicity and expression of angiogenic growth factors. This is associated with a greater rate of local recurrence and reduced survival.

Digital subtraction angiography (DSA) is the primary method for guiding TAE. The typical metric used by interventional radiologists is to count “beats of stasis” by visualizing the number of heartbeats it takes for a particle/contrast mixture to wash away under DSA. A subjective angiographic chemoembolization endpoint scale attempts to standardize the nomenclature of DSA endpoints for TAE. However, visual cues are subjective and not indicative of the physiological endpoint of the procedure, as evident in the inconsistent outcomes associated with TAE (reported tumor responses range from 15% to 85%). In fact, numerous studies have found that angiographic endpoints of TAE vary widely, and that interventional radiologists have only moderate reproducibility in classifying these endpoints when using subjective angiographic criteria.

The current standard of care has significant limitations. One of the primary causes of off-target embolization is reflux of embolic agents back along the catheter during delivery, which has been attributed to high injection pressures during the embolization phase. Since standard microcatheters rely on forward blood flow to move the embolic agent into the tumor and on systolic pressure as the packing force, if the injection pressure exceeds blood pressure, the blood flow can be reversed. This typically results in off-target embolization. The frequency of major complications for patients undergoing TAE is 9.1% per procedure, and off-target embolization accounts for 33% of these major complications.

Antireflux catheter systems attempt to prevent off-target embolization by physically occluding proximal blood vessels. In doing so, the catheter creates a low-pressure environment for the injection of beads, reportedly increasing on-target embolization. However, these catheters inherently block forward blood flow and distort visual angiographic cues that physicians rely on. Antireflux systems cause a significantly increased rate of over-embolization relative to that of standard microcatheters.

Accordingly, a way of optimally occluding a vessel such as an artery is desirable.

### SUMMARY

Disclosed herein, in one aspect, is a system including an assembly that is at least partially insertable into a patient. The assembly includes a catheter that is insertable into an artery. The catheter has an outlet. The catheter is configured to receive therethrough an embolic agent and deliver the embolic agent through the outlet. An occlusion device is coupled to the catheter. The occlusion device is configured to fully occlude the artery. A pressure sensor is configured to determine a pressure within the artery at a location proximal of the occlusion device.

In another aspect, a system includes an assembly that is at least partially insertable into a patient. The assembly includes a catheter that is insertable into an artery. The catheter has an outlet. The catheter is configured to receive therethrough an embolic agent and deliver the embolic agent through the outlet. An occlusion device is coupled to the catheter. The occlusion device is configured to fully occlude the artery. A pressure sensor is configured to determine a pressure within the artery at a location proximal of the occlusion device. The system further comprises a display and a computing device in communication with the display and the pressure sensor. The computing device has a memory and at least one processor in communication with the memory. The memory comprises instructions that, when executed by the at least one processor, cause the at least one processor to: a) receive a first pressure reading from the pressure sensor with the occlusion device in a non-occluding configuration, wherein the first pressure reading is indicative of a baseline pressure without the artery being occluded; b) receive a second pressure reading from the pressure sensor with the occlusion device in an occluding configuration, wherein the second pressure reading is indicative of the artery being fully occluded; c) receive at least one additional pressure reading from the pressure sensor; d) compare the at least one additional pressure reading to the second pressure; and e) display, on the display, a progress indicator indicating a proximity of the at least one addition pressure reading to the second pressure relative to the first pressure.

In another aspect, a method includes receiving, from a pressure sensor within an artery, a first pressure reading indicative of a baseline pressure without the artery being occluded. The artery is occluded with an occlusion device. A second pressure reading indicative of the artery being fully occluded is received from the pressure sensor within the artery proximal of the occlusion device. A threshold stop pressure is determined based on a difference between the first pressure reading and the second pressure reading.

In another aspect, a method includes receiving, from a pressure sensor within an artery, a first pressure reading indicative of a baseline pressure without the artery being occluded. The artery is occluded with an occlusion device. A second pressure reading indicative of the artery being fully occluded is received from the pressure sensor within the artery proximal of the occlusion device. At least one additional pressure reading is received from the pressure sensor. A progress indicator indicating a proximity of the at least one addition pressure reading to the second pressure relative to the first pressure is displayed on a display.

Additional advantages of the disclosed system and method will be set forth in part in the description which follows, and in part will be understood from the description, or may be learned by practice of the disclosed system and method. The advantages of the disclosed system and method will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed apparatus, system, and method and together with the description, serve to explain the principles of the disclosed apparatus, system, and method.

FIG. 1 is a schematic diagram showing an exemplary system for occluding blood vessels as disclosed herein.

FIG. 2 illustrates an exemplary assembly of the system of FIG. 1.

FIG. 3 illustrates another exemplary assembly of the system of FIG. 1.

FIG. 4 illustrates a measurement of pressure over time for embolizing a vessel.

FIG. 5 illustrates a measurement of pressure over time for a method for embolizing a vessel as disclosed herein.

FIG. 6 illustrates an exemplary computing system comprising a computing device for use with the system as disclosed herein.

### DETAILED DESCRIPTION

The disclosed system and method may be understood more readily by reference to the following detailed description of particular embodiments and the examples included therein and to the Figures and their previous and following description.

It is to be understood that the terminology used herein is for the purpose of describing particular embodiments only and is not intended to limit the scope of the present invention which will be limited only by the appended claims.

It must be noted that as used herein and in the appended claims, the singular forms “a,” “an,” and “the” include plural references unless the context clearly dictates otherwise.

Thus, for example, reference to “an embolic agent” includes one or more of such embolic agents, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Optionally, in some aspects, when values or characteristics are approximated by use of the antecedents “about,” “substantially,” or “generally,” it is contemplated that values within up to 15%, up to 10%, up to 5%, or up to 1% (above or below) of the particularly stated value or characteristic can be included within the scope of those aspects.

Unless defined otherwise, all technical and scientific terms used herein have the same meanings as commonly understood by one of skill in the art to which the disclosed apparatus, system, and method belong. Although any apparatus, systems, and methods and materials similar or equivalent to those described herein can be used in the practice or testing of the present apparatus, system, and method, the particularly useful methods, devices, systems, and materials are as described.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

As used herein, unless context dictates otherwise, “distal” refers to an end of a device positioned away from a clinician or a clinician control device, and “proximal” refers to an end of the device that is positioned toward the clinician or the clinician control device.

As used herein, when “a processor” or “at least one processor” are disclosed as performing certain steps or actions, it should be understood that such disclosure is intended to include aspects in which a single processor performs said steps or actions in any logical order, aspects in which a plurality of processors apply parallel processing to perform said steps or actions, or portions thereof, and aspects in which a plurality of processors sequentially perform said steps or actions, or portions thereof.

As used herein, a “pressure reading,” can include an instantaneous pressure reading or a plurality of measurements that are averaged or otherwise combined to provide the pressure reading (e.g., a moving average pressure reading). Thus, in some aspects, a “pressure reading” can be an instantaneous pressure reading, while in other aspects, a “pressure reading” can be a plurality of measurements that are averaged or otherwise combined to provide the pressure reading (e.g., moving average).

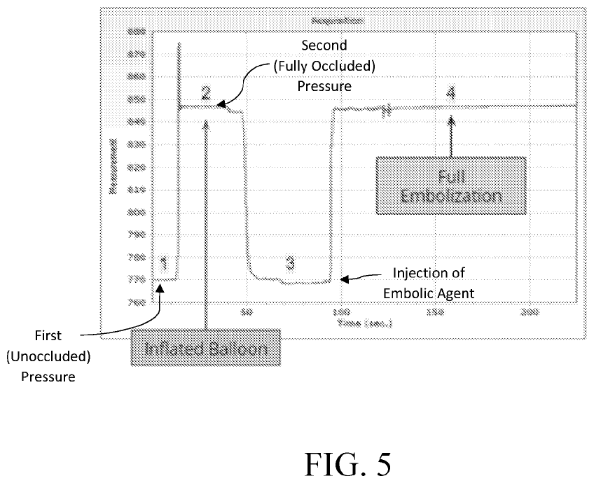

Referring to FIGS. 4 and 5, it is contemplated that fully occluding a vessel with an occlusion device can provide a fully occluded pressure that can be measured. It is further contemplated that by measuring the pressure as the blood vessel is embolized with an embolic agent, the point at which the vessel is sufficiently embolized can be determined as a pressure that is relative to the measured fully occluded pressure and the pressure without any occlusion.

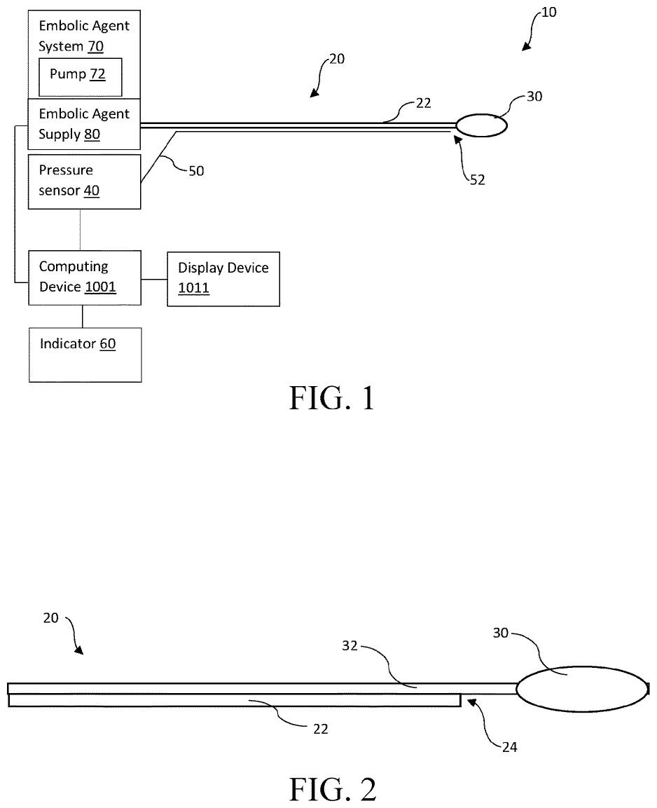

Disclosed herein and with reference to FIGS. 1-3 are embodiments of a system 10 for embolizing a blood vessel such as an artery. The system 10 can comprise an assembly 20 that is at least partially insertable into a patient. The assembly 20 can comprise a catheter 22 that is insertable into an artery. The catheter 22 can have an outlet 24. The catheter 22 can be configured to receive an embolic agent therethrough and deliver the embolic agent through the outlet 24.

The assembly 20 can further comprise an occlusion device 30 that is coupled to the catheter 22. The occlusion device 30 can be configured to fully occlude the artery. In exemplary aspects, the occlusion device 30 can be a balloon. In these aspects, the occlusion device 30 can be in communication with a conduit that is configured to deliver fluid into the balloon to inflate the balloon. In additional aspects, the occlusion device can be a vascular plug or a deployable net system such as a TRISALUS deployable net system. In still additional aspects, the occlusion device 30 can be a separate catheter that can be advanced downstream (e.g., distally) of a desired occlusion site to wedge against the walls of the blood vessel and occlude blood flow therethrough.

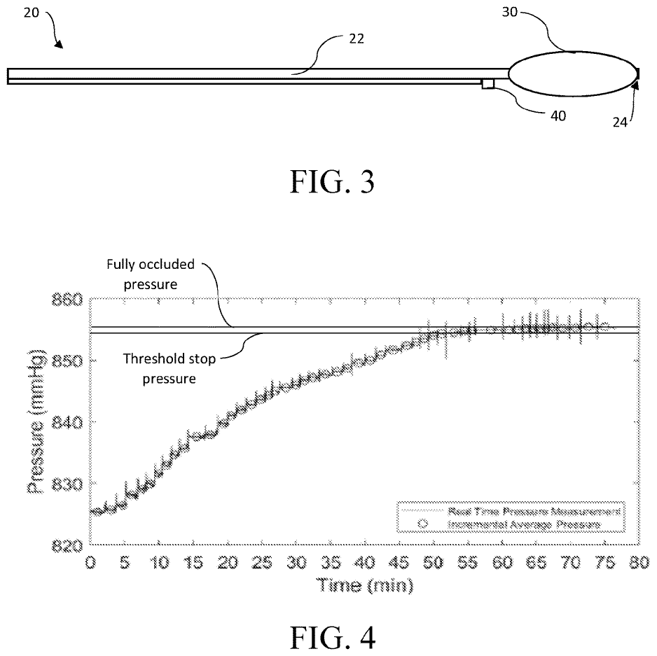

The assembly 20 can further comprise a pressure sensor 40 that is configured to determine a pressure within the artery at a location proximal of the occlusion device 30. In exemplary aspects, and as illustrated in FIG. 3, the pressure sensor 40 can be coupled to the catheter 22 and can be configured to be positioned within the artery, proximal of the occlusion device 30. In alternative aspects, and as illustrated in FIG. 1, the pressure sensor 40 can measure pressure within the artery while remaining external to the patient. For example, the assembly 20 can comprise a conduit 50 having an inlet 52 positioned proximally of the occlusion device 30, with the conduit having incompressible fluid therein that communicates pressure from the inlet 52 to the pressure sensor 40. The incompressible fluid can be, for example and without limitation, saline, sterile water, contrast, lactated ringers solution, or any suitable biocompatible fluid. The conduit 50 can communicate pressure from the inlet 52 (positioned proximate to and proximal of the occlusion device 30). The conduit 50 can be noncompliant or minimally compliant to accurately communicate pressure. That is, the conduit 50 can maintain consistent interior cross-sections when subjected to various pressures on the order of those measured. The consistent interior cross-sections can be achieved by providing a conduit with sufficient tensile strength or tensile modulus as a function of the material, conduit length, and conduit diameter.

Referring to FIG. 3, in some optional aspects, the catheter 22 can extend through the balloon, and the outlet 24 can be distal of the occlusion device 30 (e.g., a balloon). In further aspects, and as shown in FIG. 3, the catheter 22 can extend alongside a second catheter 32 that is configured to position and deploy the occlusion device 30. For example, the second catheter 32 can comprise a lumen that is configured to receive fluid therethrough for inflating and deflating the occlusion device 30.

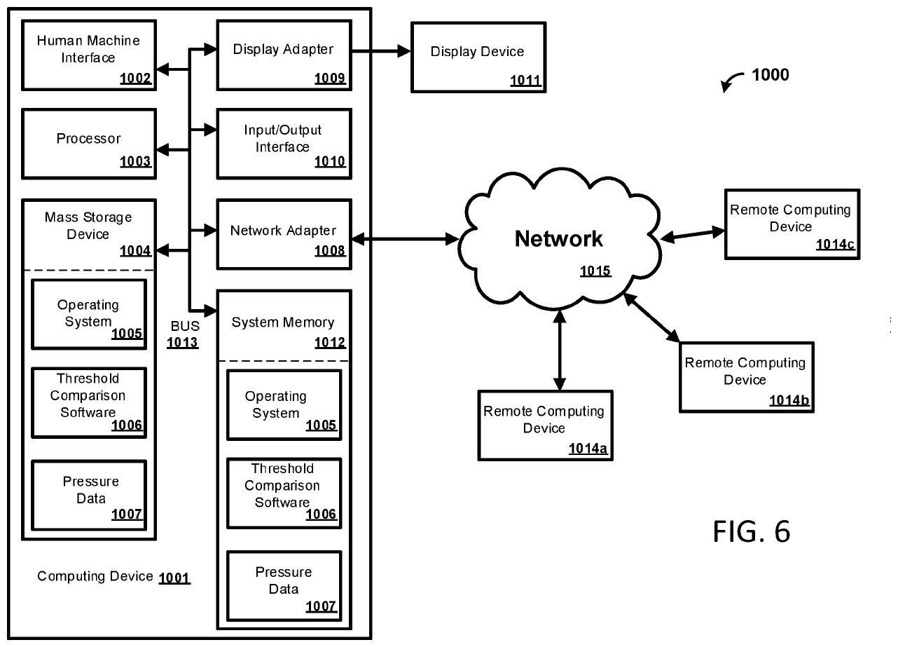

Referring also to FIG. 6, the system 10 can further comprise a computing device 1001 in communication with the pressure sensor 40, the computing device comprising a memory (e.g., mass storage device 1004) and at least one processor (e.g., processor 1003) in communication with the memory. The memory can comprise instructions that, when executed by the at least one processor, cause the at least one processor to: a) receive a first pressure reading from the pressure sensor with the occlusion device in a non-occluding configuration, wherein the first pressure reading is indicative of a baseline pressure without the artery being occluded; b) receive a second pressure reading from the pressure sensor with the occlusion device in an occluding configuration, wherein the second pressure reading is indicative of the artery being fully occluded; and c) determine a threshold stop pressure based on a difference between the first pressure reading and the second pressure reading.

The threshold stop pressure can correspond to a pressure at which the blood vessel is sufficiently embolized, yet less than a pressure consistent with off-target embolic tissue delivery (e.g., overflow of embolic agent). The threshold stop pressure (Ps) can be based on a predetermined percentage (X %) of the difference between the first pressure reading (Pl) and the second pressure reading (P2). For example, the threshold stop pressure can be calculated according to the formula: Ps=P2−X %*(P2−P1). X % can be, for example, from about 75 to about 10%, or from about 20% to about 15%, or from about 15% to about 10%, or from about 10% to about 5%, or less than 10%, less than 5, about 1%, about 2%, about 3%, about 4%, about 5%, about 6%, about 7%, about 8%, about 9%, or about 10%. It is contemplated that X % can be a function of a diameter of the vessel to be occluded, a type of vessel, or a type of procedure. For example, for embolization applications for hepatocellular carcinoma, near 100% occlusion is desirable. Partial embolization can be ideal in other clinical scenarios, such as for prostate artery embolization for the treatment of benign prostatic hyperplasia. For example, in some aspects, it can be desirable just to wean the artery, reduce blood flow, and reduce the size of the prostate, but not shut it all the way down. In these aspects, it is contemplated that the predetermined percentage can be, for example, from 25% to 90%, or about 50%, about 60%, about 70%, about 80% or about 90%.

The computing device 1001 can further be configured to detect and determine when delivery of the embolic agent should be ceased. For example, the memory can comprise instructions that, when executed by the at least one processor, cause the at least one processor to: a) receive at least one additional pressure reading from the pressure sensor; b) compare the at least one additional pressure reading to the threshold stop pressure; and c) provide a stop condition upon determining that the at least one additional pressure reading exceeds the threshold stop pressure. It is contemplated that the receiving at least one additional pressure reading can comprise continuously measuring the pressure within the vessel. In further aspects, receiving at least one additional pressure reading can comprise receiving pressure readings within the vessel at set or random intervals (e.g., every half-second, ever second, or ever two seconds).

The system 10 can further comprise an indicator 60 indicator that is configured to: a) receive the stop condition from the computing device; and b) provide an indication that the at least one additional pressure reading exceeds the threshold stop pressure. The indicator 60 can be, for example, an audible indication/alarm (e.g., a speaker) or a visual indication/alarm (e.g., a light). In further aspects, the indicator 60 can be provided on the display device 1011. That is, the computing device 1001 can cause the display device 1011 to output an indication that the pressure reading of the pressure sensor 40 has exceeded the threshold stop pressure. In further aspects, providing the stop condition can comprise causing the indicator to provide an indication that the at least one additional pressure reading exceeds the threshold stop pressure. Thus, in some aspects, receiving the stop condition from the computing device can comprise receiving a current or an electric signal that causes the indicator to indicate that the at least one additional pressure reading exceeds the threshold stop pressure.

In some optional aspects, the system 10 can comprise an embolic agent system 70 that is configured to deliver the embolic agent through the catheter 22. The embolic agent system 70 can be configured to receive the stop condition from the computing device. Upon receiving the stop condition, the embolic agent system 70 can be configured to cease delivery of the embolic agent through the catheter.

The embolic agent system 70 can comprise a pump 72 that is configured to deliver the embolic agent. The pump 72 can be, for example, a syringe pump. In some aspects, the pump can be configured to meter flow of the embolic agent. For example, is contemplated that excessive flow of the embolic agent can cause the embolic agent to spread beyond its desired delivery site. Accordingly, in some optional aspects, the pump 72 can maintain flow of the embolic agent at or below a maximum infusion rate. The maximum infusion rate can be, for example, from about 5 mL/min to about 50 mL/min (e.g., about 10 mL/min). It is further contemplated that the pump 72 can further be configured to regulate the flow rate as a function of the measured pressure in the vessel. For example, the embolic agent system 70 can reduce the maximum infusion rate as a function of proximity of the at least one additional pressure reading to the threshold stop pressure. For example, within a predetermined range of the threshold stop pressure, the computing device can be configured to cause the embolic agent system 70 to slow delivery of the embolic agent to a predetermined flow rate (e.g., optionally, from about 0.5 mL/min to about 3 mL/min). In further aspects, the computing device 1001 can continuously or intermittently vary the maximum flow rate as the measured flow rate within the vessel approaches the threshold.

The embolic agent system 70 has a maximum infusion rate, wherein the embolic agent system is configured to reduce the maximum infusion rate as a function of proximity of the at least one additional pressure reading to the threshold stop pressure.

As stated herein, in some optional aspects, the system 10 can comprise a display (e.g., display 1011) in communication with the computing device. In some aspects, the memory can comprise instructions that, when executed by the at least one processor, cause the at least one processor to: a) receive at least one additional pressure reading from the pressure sensor; b) compare the at least one additional pressure reading to the threshold stop pressure; and c) display, on the display, a progress indicator indicating a proximity of the at least one additional pressure reading to the threshold stop pressure.

The progress indicator can comprise a numerical value (e.g., a percentage), a progress bar, a change in displayed color indicative of proximity to the threshold stop pressure, or a combination thereof. Optionally, the progress indicator can be provided on a screen that overlays or is adjacent an imaging system output on a monitor. In this way, the clinician can monitor the embolization progress while viewing the imaging system output (e.g., that of an ultrasound, MRI, or CT scan). In further aspects, the progress indicator can comprise an audible indication. For example, the audible indication can comprise beeping at changing frequency depending on the embolization progress (e.g., increasing frequency as the additional pressure readings approach the threshold stop pressure). As another example, a new sound, a change in sound, or a cessation of sound can be indicative of the at least one additional pressure reading reaching the threshold stop pressure.

In various aspects, the system 10 can provide both a progress indicator and provide an output (e.g., trigger an alarm) upon exceeding a predetermined threshold.

The system 10 can further comprise a supply 80 of the embolic agent in communication with the catheter. In various optional aspects, the embolic agent can comprise at least one particle embolic (e.g., beads/microspheres), at least one solid embolic (e.g., vascular plugs or coils), at least one flowable embolic (e.g., gelfoam, epoxy, or glue), or a combination thereof.

According to further aspects, 10 system can comprise an assembly 20 as disclosed herein, a display (e.g., display device 1011), and a computing device 1001 in communication with the display and the pressure sensor of the assembly 20. The computing device 1001 can comprise a memory and at least one processor in communication with the memory, wherein the memory comprises instructions that, when executed by the at least one processor, cause the at least one processor to: a) receive a first pressure reading from the pressure sensor with the occlusion device in a non-occluding configuration, wherein the first pressure reading is indicative of a baseline pressure without the artery being occluded; b) receive a second pressure reading from the pressure sensor with the occlusion device in an occluding configuration, wherein the second pressure reading is indicative of the artery being fully occluded; c) receive at least one additional pressure reading from the pressure sensor; d) compare the at least one additional pressure reading to the second pressure; and e) display, on the display, a progress indicator indicating a proximity of the at least one addition pressure reading to the second pressure relative to the first pressure. That is, in some optional aspects, the computing device 1001 need not determine a threshold stop pressure. Rather, a clinician can determine, based on the progress indicator, when to stop and slow embolic agent delivery.

### Method of Occluding Vessels

A method of embolizing a vessel can comprise receiving, from the pressure sensor 40 within an artery, a first pressure reading indicative of a baseline pressure without the artery being occluded. The occlusion device 30 can occlude the artery. A second pressure reading indicative of the artery being fully occluded can be received from the pressure sensor 40 within the artery proximal of the occlusion device. A threshold stop pressure can be determined based on a difference between the first pressure reading and the second pressure reading.

In some aspects, an embolic agent can be delivered through a catheter to an occlusion location of the artery. At least one additional pressure reading can be received from the pressure sensor with the occlusion device in a non-occluding configuration, and with the pressure sensor proximal of the occlusion location. The at least one additional pressure reading can be compared to the threshold stop pressure. Upon determining that the at least one additional pressure reading exceeds the threshold stop pressure, delivery of the embolic agent to the artery can be ceased.

Delivery the embolic agent can comprise comprises pumping, with a pump of an embolic agent system, the embolic agent through the catheter. A stop condition can be provided to the embolic agent system upon determining that the at least one additional pressure reading exceeds the threshold stop pressure. The pump can be stopped upon providing the stop condition to the embolic agent system to cease delivery of the embolic agent to the artery.

In some aspects, an indication that the at least one additional pressure reading exceeds the threshold stop pressure can be provided. As stated herein, this can be provided as an audible indication/alarm, as a visual indication/alarm, or a change on a display.

In some aspects, an embolic agent can be delivered through a catheter to an occlusion location of the artery. At least one additional pressure reading can be received from the pressure sensor with the occlusion device in a non-occluding configuration, and with the pressure sensor proximal of the occlusion location. The at least one additional pressure reading can be compared to the threshold stop pressure. A progress indicator indicating a proximity of the at least one addition pressure reading to the threshold stop pressure can be displayed on the display.

A method of embolizing a vessel can comprise receiving, from the pressure sensor 40 within an artery, a first pressure reading indicative of a baseline pressure without the artery being occluded. The occlusion device 30 can occlude the artery. A second pressure reading indicative of the artery being fully occluded can be received from the pressure sensor 40 within the artery proximal of the occlusion device. At least one additional pressure reading can be received from the pressure sensor. A progress indicator indicating a proximity of the at least one addition pressure reading to the second pressure relative to the first pressure can be displayed on the display.

Aspects herein can be advantageous used in oncology for occluding a blood vessel for treatment of a tumor, such as, for example, hepatocellular carcinoma. However, it is contemplated that embodiments can be used for other purposes. For example, in trauma applications, a clinician can use embodiments disclosed herein to close off an actively hemorrhaging artery. Embodiments disclosed herein can be used to determine whether a hemorrhaging vessel has been adequately closed or occluded. Different embolic agents/devices, such as gelfoam, vascular coils, or vascular plugs can be used for different purposes. Various other interventional radiology applications are contemplated, such as, for example, but not limited to, peripheral artery disease, embolization of uterine fibroids, embolization for symptom reduction benign prostatic hyperplasia, genicular artery embolization for treatment of osteoarthritis of the knee, gastric artery embolization as a bariatric procedure, and arteriovenous malformations/fistulas.

In further aspects, embodiments disclosed herein can be used for measurement of flow distribution. For example, relative measures of flow distribution can be determined by acquiring and comparing occlusion pressures in different arteries. That is, an occlusion device can be inserted into different blood vessels, and pressure measurements can be taken with the occlusion device in occluding and non-occluding configurations. The absolute value of the occlusion pressure and/or the change in pressure in response to occlusion can be indicative of relative flow through a given vessel. Accordingly, relative flow distribution can be mapped. Flow distribution can be valuable in various applications, including a) probing vessels for targeting tumors for TAE therapy; and b) exploring flow states in healthy tissue for tumor tissue analysis.

### Computing Device

FIG. 6 shows a system 1000 including an exemplary configuration of a computing device 1001 for use with the system 10 (FIG. 1).

The computing device 1001 may comprise one or more processors 1003, a system memory 1012, and a bus 1013 that couples various components of the computing device 1001 including the one or more processors 1003 to the system memory 1012. In the case of multiple processors 1003, the computing device 1001 may utilize parallel computing.

The bus 1013 may comprise one or more of several possible types of bus structures, such as a memory bus, memory controller, a peripheral bus, an accelerated graphics port, and a processor or local bus using any of a variety of bus architectures.

The computing device 1001 may operate on and/or comprise a variety of computer readable media (e.g., non-transitory). Computer readable media may be any available media that is accessible by the computing device 1001 and comprises, non-transitory, volatile and/or non-volatile media, removable and non-removable media. The system memory 1012 has computer readable media in the form of volatile memory, such as random access memory (RAM), and/or non-volatile memory, such as read only memory (ROM). The system memory 1012 may store data such as pressure data 1007 and/or program modules such as operating system 1005 and threshold comparison software 1006 that are accessible to and/or are operated on by the one or more processors 1003.

The computing device 1001 may also comprise other removable/non-removable, volatile/non-volatile computer storage media. The mass storage device 1004 may provide non-volatile storage of computer code, computer readable instructions, data structures, program modules, and other data for the computing device 1001. The mass storage device 1004 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 1004. An operating system 1005 and threshold comparison software 1006 may be stored on the mass storage device 1004. One or more of the operating system 1005 and threshold comparison software 1006 (or some combination thereof) may comprise program modules and the threshold comparison software 1006. The pressure data 1007 may also be stored on the mass storage device 1004. The pressure data 1007 may be stored in any of one or more databases known in the art. The databases may be centralized or distributed across multiple locations within the network 1015.

A user may enter commands and information into the computing device 1001 using an input device. Such input devices comprise, but are not limited to, a joystick, a touchscreen display, a keyboard, a pointing device (e.g., a computer mouse, remote control), a microphone, a scanner, tactile input devices such as gloves, and other body coverings, motion sensor, speech recognition, and the like. These and other input devices may be connected to the one or more processors 1003 using a human machine interface 1002 that is coupled to the bus 1013, but may be connected by other interface and bus structures, such as a parallel port, game port, an IEEE 1394 Port (also known as a Firewire port), a serial port, network adapter 1008, and/or a universal serial bus (USB).

A display device 1011 may also be connected to the bus 1013 using an interface, such as a display adapter 1009. It is contemplated that the computing device 1001 may have more than one display adapter 1009 and the computing device 1001 may have more than one display device 1011. A display device 1011 may be a monitor, an LCD (Liquid Crystal Display), light emitting diode (LED) display, television, smart lens, smart glass, and/or a projector. In addition to the display device 1011, other output peripheral devices may comprise components such as speakers (not shown) and a printer (not shown) which may be connected to the computing device 1001 using Input/Output Interface 1010. Any step and/or result of the methods may be output (or caused to be output) in any form to an output device. Such output may be any form of visual representation, including, but not limited to, textual, graphical, animation, audio, tactile, and the like. The display 1011 and computing device 1001 may be part of one device, or separate devices.

The computing device 1001 may operate in a networked environment using logical connections to one or more remote computing devices 1014a, b, c. A remote computing device 1014a, b, c may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. Logical connections between the computing device 1001 and a remote computing device 1014a, b, c may be made using a network 1015, such as a local area network (LAN) and/or a general wide area network (WAN), or a Cloud-based network. Such network connections may be through a network adapter 1008. A network adapter 1008 may be implemented in both wired and wireless environments. Such networking environments are conventional and commonplace in dwellings, offices, enterprise-wide computer networks, intranets, and the Internet. It is contemplated that the remote computing devices 1014a, b, c can optionally have some or all of the components disclosed as being part of computing device 1001. In various further aspects, it is contemplated that some or all aspects of data processing described herein can be performed via cloud computing on one or more servers or other remote computing devices. Accordingly, at least a portion of the system 1000 can be configured with internet connectivity.

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

### Aspect 1: a System Comprising:

an assembly that is at least partially insertable into a patient, the assembly comprising:
   a catheter that is insertable into an artery, wherein the catheter has an outlet, wherein the catheter is configured to receive therethrough an embolic agent and deliver the embolic agent through the outlet; and
   an occlusion device coupled to the catheter, wherein the occlusion device is configured to fully occlude the artery; and
a pressure sensor that is configured to determine a pressure within the artery at a location proximal of the occlusion device.

Aspect 2: The system of aspect 1, further comprising a computing device in communication with the pressure sensor, the computing device comprising a memory and at least one processor in communication with the memory, wherein the memory comprises instructions that, when executed by the at least one processor, cause the at least one processor to:
receive a first pressure reading from the pressure sensor with the occlusion device in a non-occluding configuration, wherein the first pressure reading is indicative of a baseline pressure without the artery being occluded;
receive a second pressure reading from the pressure sensor with the occlusion device in an occluding configuration, wherein the second pressure reading is indicative of the artery being fully occluded; and
determine a threshold stop pressure based on a difference between the first pressure reading and the second pressure reading.

Aspect 3: The system of aspect 2, wherein the memory of the computing device comprises instructions that, when executed by the at least one processor, cause the at least one processor to:
receive at least one additional pressure reading from the pressure sensor;
compare the at least one additional pressure reading to the threshold stop pressure; and
provide a stop condition upon determining that the at least one additional pressure reading exceeds the threshold stop pressure.

Aspect 4: The system of aspect 3, further comprising an indicator, wherein providing the stop condition comprises causing the indicator to provide an indication that the at least one additional pressure reading exceeds the threshold stop pressure.

Aspect 5: The system of aspect 3 or aspect 4, further comprising an embolic agent system that is configured to deliver the embolic agent through the catheter, wherein the embolic agent system is configured to:
receive the stop condition from the computing device; and
cease, upon receiving the stop condition, delivery of the embolic agent through the catheter.

Aspect 6: The system of aspect 5, wherein the embolic agent system comprises a pump that is configured to deliver the embolic agent at or below a maximum infusion rate, wherein the maximum infusion rate is from 5 mL/min to 50 mL/min.

Aspect 7: The system of aspect 5 or aspect 6, wherein the embolic agent system has a maximum infusion rate, wherein the embolic agent system is configured to reduce the maximum infusion rate as a function of proximity of the at least one additional pressure reading to the threshold stop pressure.

Aspect 8: The system of any one of aspects 2-7, further comprising a display in communication with the computing device, wherein the memory comprises instructions that, when executed by the at least one processor, cause the at least one processor to:
receive at least one additional pressure reading from the pressure sensor;
compare the at least one additional pressure reading to the threshold stop pressure; and
display, on the display, a progress indicator indicating a proximity of the at least one addition pressure reading to the threshold stop pressure.

Aspect 9: The system of any one of the preceding aspects, wherein the threshold stop pressure (Pₛ) is based on a predetermined percentage (X %) of the difference between the first pressure reading (P1) and the second pressure reading (P2), wherein the threshold stop pressure is calculated according to the formula: Pₛ=P2−X %*(P2−P1).

Aspect 10: The system of any one of the preceding aspects, wherein the predetermined percentage is 10% or less.

Aspect 11: The system of any one of the preceding aspects, wherein the predetermined percentage is 5% or less.

Aspect 12: The system of any one of the preceding aspects, further comprising a supply of the embolic agent in communication with the catheter.

Aspect 13: The system of aspect 12, wherein the embolic agent comprises at least one particle embolics, at least one solid embolic, at least one flowable embolic, or a combination thereof.

Aspect 14: The system of any one of the preceding aspects, wherein the occlusion device is a balloon.

Aspect 15: The system of aspect 14, wherein the catheter extends through the balloon.

Aspect 16: The system of any one of the preceding aspects, wherein the pressure sensor is coupled to the catheter and positioned proximally of the occlusion device.

Aspect 17: The system of any one of aspects 1-15, wherein the assembly comprises a conduit having an inlet positioned proximally of the occlusion device, wherein the conduit contains incompressible fluid therein that communicates pressure from the inlet to the pressure sensor.

Aspect 18: A system comprising:
an assembly that is at least partially insertable into a patient, the assembly comprising:
   a catheter that is insertable into an artery, wherein the catheter has an outlet, wherein the catheter is configured to receive therethrough an embolic agent and deliver the embolic agent through the outlet;
   an occlusion device coupled to the catheter, wherein the occlusion device is configured to fully occlude the artery;
   a pressure sensor that is configured to determine a pressure within the artery at a location proximal of the occlusion device;
a display; and
a computing device in communication with the display and the pressure sensor, the computing device comprising a memory and at least one processor in communication with the memory, wherein the memory comprises instructions that, when executed by the at least one processor, cause the at least one processor to:
   receive a first pressure reading from the pressure sensor with the occlusion device in a non-occluding configuration, wherein the first pressure reading is indicative of a baseline pressure without the artery being occluded;
   receive a second pressure reading from the pressure sensor with the occlusion device in an occluding configuration, wherein the second pressure reading is indicative of the artery being fully occluded;
   receive at least one additional pressure reading from the pressure sensor; compare the at least one additional pressure reading to the second pressure; and
   display, on the display, a progress indicator indicating a proximity of the at least one addition pressure reading to the second pressure relative to the first pressure.

Aspect 19: A method comprising:
receiving, from a pressure sensor within an artery, a first pressure reading indicative of a baseline pressure without the artery being occluded;
occluding, with an occlusion device, the artery;
receiving, from the pressure sensor within the artery proximal of the occlusion device, a second pressure reading indicative of the artery being fully occluded; and
determining a threshold stop pressure based on a difference between the first pressure reading and the second pressure reading.

Aspect 20: The method of aspect 19, further comprising:
delivering, to the artery through a catheter, an embolic agent to an occlusion location;
receiving, with the occlusion device in a non-occluding configuration, and with the pressure sensor proximal of the occlusion location, at least one additional pressure reading from the pressure sensor;
comparing the at least one additional pressure reading to the threshold stop pressure; and
ceasing, upon determining that the at least one additional pressure reading exceeds the threshold stop pressure, delivery of the embolic agent to the artery.

Aspect 21: The method of aspect 20, wherein delivery the embolic agent comprises pumping, with a pump of an embolic agent system, the embolic agent through the catheter, wherein the method further comprises providing, to the embolic agent system, a stop condition upon determining that the at least one additional pressure reading exceeds the threshold stop pressure,
wherein ceasing delivery of the embolic agent to the artery comprises causing the pump to stop pumping upon providing the stop condition to the embolic agent system.

Aspect 22: The method of aspect 21, wherein the pump is configured to deliver the embolic agent at or below a maximum infusion rate.

Aspect 23: The method of aspect 19, further comprising: providing an indication that the at least one additional pressure reading exceeds the threshold stop pressure.

Aspect 24: The method of aspect 19, further comprising:
delivering, to the artery through a catheter, an embolic agent to an occlusion location;
receiving, with the occlusion device in a non-occluding configuration, and with the pressure sensor proximal of the occlusion location, at least one additional pressure reading from the pressure sensor;
comparing the at least one additional pressure reading to the threshold stop pressure; and
displaying, on a display, a progress indicator indicating a proximity of the at least one addition pressure reading to the threshold stop pressure.

Aspect 25: The method of any one of aspects 19-24, wherein the threshold stop pressure (Pₛ) is based on a predetermined percentage (X %) of the difference between the first pressure reading (P1) and the second pressure reading (P2), wherein the threshold stop pressure is calculated according to the formula: Pₛ=P2−X %*(P2−P1).

Aspect 26: The method of any one of aspects 19-25, wherein the predetermined percentage is 10% or less.

Aspect 27: The method of any one of aspects 19-26, wherein the predetermined percentage is 5% or less.

Aspect 28: The method of any one of aspects 20-27, wherein the embolic agent comprises at least one particle embolic, at least one solid embolic, at least one flowable embolic, or a combination thereof.

Aspect 29: The method of any one of aspects 19-28, wherein the occlusion device is a balloon.

Aspect 30: A method comprising:
receiving, from a pressure sensor within an artery, a first pressure reading indicative of a baseline pressure without the artery being occluded;
occluding, with an occlusion device, the artery;
receiving, from the pressure sensor within the artery proximal of the occlusion device, a second pressure reading indicative of the artery being fully occluded; and
receiving at least one additional pressure reading from the pressure sensor; and
displaying, on a display, a progress indicator indicating a proximity of the at least one addition pressure reading to the second pressure relative to the first pressure.

Those skilled in the art will recognize, or be able to ascertain using no more than routine experimentation, many equivalents to the specific embodiments of the method and compositions described herein. Such equivalents are intended to be encompassed by the following claims.

1. A system comprising:
an assembly that is at least partially insertable into a patient, the assembly comprising:
a catheter that is insertable into an artery, wherein the catheter has an outlet, wherein the catheter is configured to receive therethrough an embolic agent and deliver the embolic agent through the outlet; and
an occlusion device coupled to the catheter, wherein the occlusion device is configured to fully occlude the artery; and
a pressure sensor that is configured to determine a pressure within the artery at a location proximal of the occlusion device.

2. The system of claim 1, further comprising a computing device in communication with the pressure sensor, the computing device comprising a memory and at least one processor in communication with the memory, wherein the memory comprises instructions that, when executed by the at least one processor, cause the at least one processor to:
receive a first pressure reading from the pressure sensor with the occlusion device in a non-occluding configuration, wherein the first pressure reading is indicative of a baseline pressure without the artery being occluded;
receive a second pressure reading from the pressure sensor with the occlusion device in an occluding configuration, wherein the second pressure reading is indicative of the artery being fully occluded; and
determine a threshold stop pressure based on a difference between the first pressure reading and the second pressure reading.

3. The system of claim 2, wherein the memory of the computing device comprises instructions that, when executed by the at least one processor, cause the at least one processor to:
receive at least one additional pressure reading from the pressure sensor;
compare the at least one additional pressure reading to the threshold stop pressure; and
provide a stop condition upon determining that the at least one additional pressure reading exceeds the threshold stop pressure.

4. The system of claim 3, further comprising an indicator, wherein providing the stop condition comprises causing the indicator to provide an indication that the at least one additional pressure reading exceeds the threshold stop pressure.

5. The system of claim 3, further comprising an embolic agent system that is configured to deliver the embolic agent through the catheter, wherein the embolic agent system is configured to:
receive the stop condition from the computing device; and
cease, upon receiving the stop condition, delivery of the embolic agent through the catheter.

6. (canceled)

7. (canceled)

8. The system of claim 2, further comprising a display in communication with the computing device, wherein the memory comprises instructions that, when executed by the at least one processor, cause the at least one processor to:
receive at least one additional pressure reading from the pressure sensor;
compare the at least one additional pressure reading to the threshold stop pressure; and
display, on the display, a progress indicator indicating a proximity of the at least one addition pressure reading to the threshold stop pressure.

9. The system of claim 1, wherein the threshold stop pressure (Pₛ) is based on a predetermined percentage (X %) of the difference between the first pressure reading (P1) and the second pressure reading (P2), wherein the threshold stop pressure is calculated according to the formula: Pₛ=P2−X %*(P2−P1).

10. The system of claim 9, wherein the predetermined percentage is 10% or less.

11. (canceled)

12. The system of claim 1, further comprising a supply of the embolic agent in communication with the catheter.

13. (canceled)

14. The system of claim 1, wherein the occlusion device is a balloon.

15. The system of claim 14, wherein the catheter extends through the balloon.

16. The system of claim 1, wherein the pressure sensor is coupled to the catheter and positioned proximally of the occlusion device.

17. The system of claim 1, wherein the assembly comprises a conduit having an inlet positioned proximally of the occlusion device, wherein the conduit contains incompressible fluid therein that communicates pressure from the inlet to the pressure sensor.

18. A system comprising:
an assembly that is at least partially insertable into a patient, the assembly comprising:
a catheter that is insertable into an artery, wherein the catheter has an outlet, wherein the catheter is configured to receive therethrough an embolic agent and deliver the embolic agent through the outlet;
an occlusion device coupled to the catheter, wherein the occlusion device is configured to fully occlude the artery;
a pressure sensor that is configured to determine a pressure within the artery at a location proximal of the occlusion device;
a display; and
a computing device in communication with the display and the pressure sensor, the computing device comprising a memory and at least one processor in communication with the memory, wherein the memory comprises instructions that, when executed by the at least one processor, cause the at least one processor to:
receive a first pressure reading from the pressure sensor with the occlusion device in a non-occluding configuration, wherein the first pressure reading is indicative of a baseline pressure without the artery being occluded;
receive a second pressure reading from the pressure sensor with the occlusion device in an occluding configuration, wherein the second pressure reading is indicative of the artery being fully occluded;
receive at least one additional pressure reading from the pressure sensor;
compare the at least one additional pressure reading to the second pressure; and
display, on the display, a progress indicator indicating a proximity of the at least one addition pressure reading to the second pressure relative to the first pressure.

19. A method comprising:
receiving, from a pressure sensor within an artery, a first pressure reading indicative of a baseline pressure without the artery being occluded;
occluding, with an occlusion device, the artery;
receiving, from the pressure sensor within the artery proximal of the occlusion device, a second pressure reading indicative of the artery being fully occluded; and
determining a threshold stop pressure based on a difference between the first pressure reading and the second pressure reading.

20. The method of claim 19, further comprising:
delivering, to the artery through a catheter, an embolic agent to an occlusion location;
receiving, with the occlusion device in a non-occluding configuration, and with the pressure sensor proximal of the occlusion location, at least one additional pressure reading from the pressure sensor;
comparing the at least one additional pressure reading to the threshold stop pressure; and
ceasing, upon determining that the at least one additional pressure reading exceeds the threshold stop pressure, delivery of the embolic agent to the artery.

21. The method of claim 20, wherein delivering the embolic agent comprises pumping, with a pump of an embolic agent system, the embolic agent through the catheter, wherein the method further comprises providing, to the embolic agent system, a stop condition upon determining that the at least one additional pressure reading exceeds the threshold stop pressure, wherein ceasing delivery of the embolic agent to the artery comprises causing the pump to stop pumping upon providing the stop condition to the embolic agent system.

22. The method of claim 21, wherein the pump is configured to deliver the embolic agent at or below a maximum infusion rate.

23. The method of claim 19, further comprising: providing an indication that the at least one additional pressure reading exceeds the threshold stop pressure.

24. The method of claim 19, further comprising:
delivering, to the artery through a catheter, an embolic agent to an occlusion location;
receiving, with the occlusion device in a non-occluding configuration, and with the pressure sensor proximal of the occlusion location, at least one additional pressure reading from the pressure sensor;
comparing the at least one additional pressure reading to the threshold stop pressure; and
displaying, on a display, a progress indicator indicating a proximity of the at least one addition pressure reading to the threshold stop pressure.

25. (canceled)

26. (canceled)

27. (canceled)

28. (canceled)

29. (canceled)

30. (canceled)
